(19) United States
(12) Patent Application Publication
(10) Pub. No.: US 2012/0100221 A1
(43) Pub. Date: Apr. 26, 2012

(54) PHARMACEUTICAL COMPOSITIONS CONTAINING A COMBINATION OF AN ANTIHISTAMINE AND A DECONGESTANT

(75) Inventors: Anuj Kumar Fanda, Uttar Pradesh (IN); Kumaravel Vivek, Tamil Nadu (IN); Romi Barat Singh, Uttar Pradesh (IN)

(73) Assignee: RANBAXY LABORATORIES LIMITED, New Delhi, Delhi (IN)

(21) Appl. No.: 13/375,369

(22) PCT Filed: May 31, 2010
(86) PCT No.: PCT/IB10/52425

§ 371 Date: Jan. 6, 2012

(30) Foreign Application Priority Data
Jun. 2, 2009 (IN) 1127/DEL/2009

(51) Int. Cl. A61K 9/14; A61P 11/02; A61P 37/08; A61K 31/137; A61K 31/445

(52) U.S. Cl. 424/495; 514/653; 514/317; 424/494; 424/497; 424/498

(57) ABSTRACT

This invention relates to a layered pharmaceutical composition comprising a combination of an antihistamine and a decongestant.

### TECHNICAL FIELD OF THE INVENTION

This invention relates to a layered pharmaceutical composition comprising a combination of an antihistamine and a decongestant.

### BACKGROUND OF THE INVENTION

A decongestant is commonly administered orally in combination with an antihistamine for relieving nasal congestion associated with allergic rhinitis. Based on their biological half life, it is quite apparent that when the decongestant is pseudoephedrine or its pharmaceutically acceptable salt, and the antihistamine is a long-acting antihistamine (e.g., fexofenadine), then the dosage form should preferably be designed such that the long acting antihistamine is released in a conventional manner and pseudoephedrine is released at a controlled rate, such that the pharmaceutical composition is suitable for twice-daily or once-daily administration.

Such a combination of decongestants and antihistamines offers more complete relief of rhinitis symptoms than therapy with either component alone. Commercially these combinations are available as: (i) ALLEGRA-D® 24 Hour Extended-Release Tablets (manufactured by Sanofi-Aventis, US) for once-daily oral administration containing 180 mg of fexofenadine hydrochloride and 240 mg of pseudoephedrine hydrochloride; and (ii) ALLEGRA-D® 12 Hour Extended-Release Tablets (manufactured by Sanofi-Aventis, US) for twice-daily oral administration containing 60 mg of fexofenadine hydrochloride and 120 mg of pseudoephedrine hydrochloride.

U.S. Pat. No. 6,613,357 describes an osmotic device containing controlled release pseudoephedrine in a core in combination with a rapid release H1 antagonist in an external coat. U.S. Pat. No. 6,039,974 discloses a combination of piperidinoalkanol and decongestant in the form of a bilayer tablet. U.S. Pat. No. 6,004,582 describes a multi-layered osmotic device and U.S. Pat. No. 6,537,573 discloses a dosage form containing cetirizine as an intermediate release component and pseudoephedrine as a controlled release component.

Without controlled release of the drugs, the schedule for administering a combination of piperidinoalkanol compound and decongestant is typically three or four doses per day. In order to provide a once-daily or twice-daily dosage form a formulation providing a relatively immediate release of the piperidinoalkanol with an extended release of the decongestant is desirable.

### SUMMARY OF THE INVENTION

In one general aspect there is provided a layered composition of an antihistamine and a decongestant. The layered composition includes:

(i) an antihistamine layer, which includes:
(a) therapeutically effective amount of an antihistamine or its pharmaceutically effective salts; and
(b) one or more pharmaceutically acceptable excipients;

(ii) a decongestant layer, which includes:
(a) a core, which includes therapeutically effective amount of decongestant or its pharmaceutically effective salts;
(b) a coating over the core, which includes one or more rate-controlling polymers; and
(c) one or more pharmaceutically acceptable excipients.

Embodiments of the present invention may include one or more of the following features. For example, the core can be embedded in a carrier matrix.

The antihistamine may be an H1 antagonist. For example, the antihistamine may be fexofenadine, loratadine, cetirizine, terfenadine, acrivastine, astemizole, and its pharmaceutically acceptable salts, solvates, polymorphs, enantiomers, single isomer, or mixtures thereof. The decongestant may be pseudoephedrine, oxymetazoline, phenylephrine, xylometazoline, and its pharmaceutically acceptable salts, solvates, polymorphs, enantiomers, single isomer, or mixtures thereof. In one embodiment, the antihistamine is fexofenadine and the decongestant is pseudoephedrine.

The decongestant layer may further include one or more cushioning agents. The cushioning agent may be one or more of waxes, fats, lipids, polyethylene glycols, polyoxyethylenes, or gums or mixture thereof.

The rate-controlling polymers used to provide coating over decongestant core may be one or more of hydrophilic polymers, hydrophobic polymers or combinations thereof. The hydrophilic rate controlling polymer may be one or more of cellulose derivatives, polyvinylpyrrolidone, polyvinyl acetate, copolymer of vinylpyrrolidone and vinyl acetate, polysaccharides, polyalkylene glycols, starch, gums and derivatives or mixtures thereof. The hydrophobic rate controlling polymer may be one or more of ethyl cellulose, cellulose acetate, cellulose acetate butyrate, hydroxypropyl methylcellulose phthalate, poly (alkyl)methacrylate, and copolymers of acrylic or methacrylic acid esters, waxes, shellac or hydrogenated vegetable oils or mixtures thereof.

The excipients used in the present invention may be one or more of fillers, binders, disintegrants, lubricants, glidants, colorants or flavoring agents or mixtures thereof.

In another general aspect, there is provided a process for preparing the layered composition. The process includes the steps of:

(i) providing an antihistamine layer by:
(a) preparing granules, which include an antihistamine or its pharmaceutically effective salts with one or more pharmaceutically acceptable excipients; and
(b) combining the granules of antihistamine with one or more pharmaceutically acceptable excipients;

(ii) providing a decongestant layer by:
(a) preparing cores, which include a decongestant or its pharmaceutically effective salts;
(b) coating the cores with one or more rate-controlling polymers; and
(c) combining the coated cores of decongestant with one or more pharmaceutically acceptable excipients; and

(iii) compressing the antihistamine layer and the decongestant layer into layered composition.

In another general aspect the present invention provides for a process for preparing the layered composition. The process includes the steps of:

(i) providing an antihistamine layer by:
(a) preparing a solution, suspension or dispersion, which includes an antihistamine or its pharmaceutically effective salts with one or more pharmaceutically acceptable excipients;

(ii) providing a decongestant layer by:
(a) preparing cores, which include a decongestant or its pharmaceutically effective salts;
(b) optionally coating the cores with a seal coating;
(c) coating the cores with a coating, which includes one or more rate-controlling polymers;

(iii) layering the antihistamine layer over the decongestant layer to form a layered composition.

In a final general aspect, there is provided a method for treating nasal congestion or allergy-related disorders by administering a layered composition of an antihistamine and a decongestant, wherein the composition includes:

(i) an antihistamine layer, which includes:
(a) therapeutically effective amount of an antihistamine or its pharmaceutically effective salts; and
(b) one or more pharmaceutically acceptable excipients;

(ii) a decongestant layer, which includes:
(a) a core, which includes therapeutically effective amount of decongestant or its pharmaceutically effective salts;
(b) a coating over the core, which includes one or more rate-controlling polymers; and
(c) one or more pharmaceutically acceptable excipients.

### DETAILED DESCRIPTION OF THE INVENTION

The object of the present invention is to provide a pharmaceutical composition in the form of layered composition which includes an immediate release layer of an antihistamine and a controlled release layer of a decongestant.

The layered composition of an antihistamine and a decongestant includes:

(i) an antihistamine layer includes:
(a) therapeutically effective amount of an antihistamine or its pharmaceutically effective salts; and
(b) one or more pharmaceutically acceptable excipients;

(ii) a decongestant layer includes:
(a) a core, which includes a therapeutically effective amount of decongestant or its pharmaceutically effective salts;
(b) a coating over the core, which includes one or more rate-controlling polymers; and
(c) one or more pharmaceutically acceptable excipients.

Further, the present invention provides a process for preparing the layered composition. The process includes the steps of:

(i) providing an antihistamine layer by:
(a) preparing the granules, which includes an antihistamine or its pharmaceutically effective salts with one or more pharmaceutically acceptable excipients; and
(b) combining the granules of antihistamine with one or more pharmaceutically acceptable excipients;

(ii) providing a decongestant layer by:
(a) preparing the cores, which includes a decongestant or its pharmaceutically effective salts;
(b) coating the cores with comprising one or more rate-controlling polymers; and
(c) combining the coated cores of decongestant with one or more pharmaceutically acceptable excipients; and

(iii) compressing the antihistamine layer and the decongestant layer into layered composition.

The present invention also provides a second process for preparing the layered composition. The process includes the steps of:

(i) providing an antihistamine layer by:
(a) preparing the solution, suspension or dispersion, which comprises an antihistamine or its pharmaceutically effective salts with one or more pharmaceutically acceptable excipients;

(ii) providing a decongestant layer by:
(a) preparing the cores, which comprises a decongestant or its pharmaceutically effective salts;
(b) optionally coating the cores with seal coating;
(c) coating the cores with coating, which comprises one or more rate-controlling polymers;

(iii) layering the antihistamine layer over the decongestant layer to form a layered composition.

The antihistamine granules present in the antihistamine layer are prepared in accordance with any pharmaceutically acceptable technique that achieves uniform blending, e.g. dry blending, wet granulation and dry granulation.

In the dry blending step, a suitable antihistamine and the pharmaceutically acceptable excipients are blended together and compressed. The wet granulation method involves mixing an antihistamine and pharmaceutically acceptable excipients, with a solution or dispersion of a wet binder and then granulating into desired size granules. The granules may also be prepared by the techniques known in the field of art, for example, simple granulation, followed by sieving; drug layering; extrusion and marumerization or spheronization; rotogranulation; pelletization; micropelletization, etc. These steps may be carried out in the conventional manner. The dry granulation process includes compacting antihistamine with one or more pharmaceutically acceptable excipient(s) by using any suitable apparatus, for example, roller compactor such as a chilsonator or drop roller; or a conventional tablet press. The techniques may involve roller compaction or slugging and sizing the compacts into granules by milling.

The decongestant cores may be prepared as aggregated particles, pellets, mini tablets, tablets, beads or granules. The cores may be prepared in accordance with any of the conventional procedures known in the field of art, for example, simple granulation followed by sieving; extrusion and marumerization or spheronization; rotogranulation; pelletization; micropelletization; coating etc. These steps may be carried out in the conventional manner.

The decongestant cores may also be prepared by layering or depositing decongestant or its effective salts with one or more hydrophilic polymer(s), onto inert cores. Layering decongestant-containing solution or dispersion onto inert cores can be achieved by various techniques including dipping, spraying and layering. Layering can also be achieved by spraying using fluidized bed technology with Wurster, top spray or side spray techniques.

The inert cores used to prepare decongestant cores may be pellets, beads, spheres, particles or seeds that may be water-soluble, water swellable, or water-insoluble; and organic or inorganic, or mixtures thereof. The size of cores generally ranges from about 20 μm to about 5000 μm.

The water-soluble or water swellable inert cores may be one or more of soluble cores such as sugar spheres having sugars like dextrose, lactose, anhydrous lactose, spray-dried lactose, lactose monohydrate, mannitol, starches, sorbitol, or sucrose. The commercially available inert core materials may also be utilized, which is one or more of sugar sphere, non-pareil seed, and celphere.

Water-insoluble inert cores may include one or more of glass particles/beads or silicon dioxide, calcium phosphate dihydrate, dicalcium phosphate, calcium sulfate dihydrate, microcrystalline cellulose (e.g., Avicel™), silicified microcrystalline cellulose (e.g., Prosolv™), cellulose derivatives, powdered cellulose (e.g., Elcema™ G 250 manufactured by Degussa), or mixtures thereof.

Suitable examples of hydrophilic polymer(s) used in layering or depositing the decongestant onto the inert cores include cellulose ethers, such as hydroxypropyl methylcellulose, hydroxypropylcellulose, or other water soluble or swellable polymers, such as sodium carboxymethyl cellulose, xanthan gum, acacia, tragacanth gum, guar gum, karaya gum, alginates, gelatin, albumin and the like. The hydrophilic polymers, used may also be polyacrylate polymers such as homopolymers based on acrylic acid crosslinked with allyl sucrose or allyl pentaerythritol, or copolymer based on acrylic acid and long chain (C₁₀-C₃₀) alkyl acrylates and cross-linked with allylpentaerythritol.

The polymers used to provide seal coating over the decongestant core may include one or more of a hydrophilic polymer. Examples include hydroxypropyl cellulose, hydroxypropylisopropylcellulose, hydroxypropylmethylcellulose, methoxypropyl cellulose, hydroxypropylpentylcellulose, hydroxypropylhexylcellulose and any mixtures thereof.

The rate-controlling polymers used to provide coating over the decongestant core may include one or more of hydrophilic polymers, hydrophobic polymers, or combinations thereof.

Suitable examples of hydrophilic rate controlling polymers include cellulose derivatives such as hydroxypropylcellulose, hydroxypropylmethylcellulose, hydroxyethylcellulose, hydroxymethylcellulose, carboxymethylcellulose, methylcellulose, sodium carboxy methylcellulose or combinations thereof; polyvinylpyrrolidone, polyvinyl acetate, copolymer of vinylpyrrolidone and vinyl acetate, polysaccharides, polyalkylene glycols, starch, gums and derivatives; or mixtures thereof.

Suitable examples of hydrophobic rate controlling polymers include ethyl cellulose, cellulose acetate, cellulose acetate butyrate, hydroxypropyl methylcellulose phthalate, poly (alkyl)methacrylate, and copolymers of acrylic or methacrylic acid esters, waxes, shellac and hydrogenated vegetable oils.

The rate controlling polymers and the seal coating polymers may additionally include, plasticizers selected from propylene glycol, triethylene glycol, oleic acid, ethyleneglycol monoleate, triethyl citrate, triacetin, diethyl phthalate, glyceryl monostearate, dibutyl sebacate, acetyl triethyl citrate, acetyl tributyl citrate, castor oil or mixtures thereof.

Example of solvents used for preparing a solution/dispersion of rate controlling polymers and seal coating polymers to coat the decongestant cores include methylene chloride, isopropyl alcohol, acetone, methanol, ethanol, water or mixtures thereof.

The rate controlling polymers and seal coating polymers may be coated using a conventional coating pan, a spray coater, a rotating perforated pan, or an automated system, such as a centrifugal fluidizing (CF) granulator, a fluidized bed process, or any other suitably automated coating equipment.

The cores of decongestant may be prepared by embedding in a carrier matrix comprising the rate controlling polymers and one or more pharmaceutically acceptable excipients that may act in one or more capacities as fillers, binders, disintegrants, lubricants and glidants.

The decongestant layer may additionally include one or more cushioning agents. Cushioning agents are agents that provide flexibility to coated decongestant cores such that when compressed into a tablet, the coated decongestant cores substantially retain their structural integrity and do not rupture in a significant way or as a significant fraction of the decongestant cores.

The cushioning agents may be layered over the decongestant cores or may present along the decongestant cores or the carrier matrix. Layering of cushioning agent can be done by using a conventional coating pan, a spray coater, a rotating perforated pan, or an automated system, such as a centrifugal fluidizing (CF) granulator, a fluidized bed process, or any other suitably automated coating equipment.

Examples of cushioning agents include waxes, fats, lipids, polyethylene glycols, polyoxyethylenes, and gums. Examples of waxes include carnuba wax, bees wax, sperm whale wax, etc. Examples of fats and lipids include lecithin, hydrogenated vegetable oils, hydrogenated castor oil, hydrogenated sesame oil, etc., Examples of gums include gum arabica, xanthan gum, gum acacia, etc.

The pharmaceutically acceptable excipients used in the antihistamine layer or in the decongestant layer or in the carrier matrix may include fillers, binders, disintegrants, lubricants, glidants, colorants and flavoring agents.

Suitable examples of fillers include corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, sorbitol, starch, starch pregelatinized, sucrose, or mixtures thereof.

Examples of binders include methyl cellulose, hydroxypropyl cellulose, hydroxypropyl methylcellulose, polyvinylpyrrolidone, poloxamer, gelatin, gum Arabic, ethyl cellulose, polyvinyl alcohol, pullutan, pregelatinized starch, agar, tragacanth, sodium alginate, propylene glycol, or mixtures thereof.

Examples of disintegrants include starch, croscarmellose sodium, crospovidone, sodium starch glycolate or mixtures thereof.

Examples of lubricants and glidants include colloidal anhydrous silica, stearic acid, magnesium stearate, calcium stearate, talc, hydrogenated castor oil, sucrose esters of fatty acids, microcrystalline wax, yellow beeswax, white beeswax, or mixtures thereof.

The coloring agents of the present invention may be selected from any FDA approved color for oral use.

The antihistamine granules and the decongestant granules with one or more pharmaceutically acceptable excipients may be compressed into layered composition e.g. as layered tablet, compression-coated tablet or inlay tablet.

A layered tablet is a tablet which is made up of two or more distinct layers or discrete zones of layers compressed together with the individual layers lying one on top of another or adjacent to the other. Such conventional layered tablets are generally prepared by compressing a granulation onto a previously compressed granulation. The operation may be repeated to produce multilayered tablets of more than two layers. A layered tablet has at least two layers or discrete zones one of which is made from antihistamine layer and another of which is made from decongestant layer.

A compression-coated tablet is a tablet which is made up of an inner core and one or more outer coats wherein the inner core is completely surrounded by the outer coat or coats. These tablets have at least two discrete zones of granulation compressed together, i.e., an inner core zone and an outer coat zone. Such tablets are prepared by feeding a previously compressed inner core into a special tableting machine and compressing one or more other granulation coats around the preformed inner core.

A variation of the compression-coated tablet is the inlay tablet, also referred to as a dot, or bull's-eye tablet. Instead of an inner core zone being completely surrounded by an outer coat, one surface of the zone corresponding to an inner core zone is exposed. These tablets have at least two discrete zones of granulation compressed together. The preparation of inlay tablets is similar to the preparation of compression-coated tablets except that a surface of coating is eliminated.

A barrier layer may be included between the antihistamine layer and the decongestant layer in case of any instability between the two actives. The barrier layer is a physical barrier around one or both of the actives. In one or the embodiments, the barrier is a coating around at least one of the two actives.

The layered composition may optionally be coated with one or more layers comprising film forming agents and/or pharmaceutically acceptable excipients.

Example of film forming agents include ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate; waxes, such as polyethylene glycol; methacrylic acid polymers such as Eudragit® RL and RS; or mixture thereof. Alternatively, commercially available coating compositions including film-forming polymers marketed under various trade names, such as Opadry® may also be used for coating.

The film forming agents may be applied as solution/dispersion of coating ingredients using any conventional technique known in the prior art such as spray coating in a conventional coating pan or fluidized bed processor or dip coating.

Example of solvents used for preparing a solution/dispersion of the coating ingredients include methylene chloride, isopropyl alcohol, acetone, methanol, ethanol, water or mixtures thereof.

The present invention is illustrated below by reference to the following example. However, one skilled in the art will appreciate that the specific methods and results discussed are merely illustrative of the invention, and not to be construed as limiting the invention.

### Example 1

### I. Preparation of Pseudoephedrine Layer

| | |
|---|---|
| | Percent w/w of |
| Ingredients | tablet |

Core

| | |
|---|---|
| Microcrystalline cellulose spheres | 6.22 |
| Pseudoephedrine hydrochloride | 14.94 |
| Hydroxypropyl methyl cellulose | 1.49 |
| Purified water | Q.S |

Rate-controlling polymer coating

| | |
|---|---|
| Ethyl cellulose | 5.21 |
| Acetyl tributyl citrate | 0.52 |
| Isopropyl alcohol | Q.S |
| Purified water | Q.S |

Cushioning agent coating

| | |
|---|---|
| Polyethylene glycol | 2.84 |
| Isopropyl alcohol | Q.S |
| Purified water | Q.S |

Extra granular ingredients

| | |
|---|---|
| Polyethylene glycol 6000 | 3.98 |
| Polyvinylpyrrolidone | 2.49 |
| Microcrystalline cellulose | 27.34 |
| Colloidal silicon dioxide | 0.62 |
| Magnesium stearate | 0.31 |

1. Pseudoephedrine hydrochloride and hydroxypropyl methyl cellulose were dissolved in purified water and layered over microcrystalline cellulose spheres.
2. The cores of step 1 were coated with the rate-controlling polymer coating composition.
3. The cores of step 2 were coated with the cushioning agent coating composition.
4. The cores of step 3 were mixed with the extra granular ingredients to form pseudoephedrine blend.

### II. Preparation of Fexofenadine Layer

| | |
|---|---|
| | Percent w/w of |
| Ingredients | tablet |

Intra granular ingredients

| | |
|---|---|
| Fexofenadine hydrochloride | 11.20 |
| Cross linked Polyvinylpyrrolidone | 0.93 |
| Polyvinylpyrrolidone | 1.24 |
| Microcrystalline cellulose | 9.96 |
| Colloidal silicon dioxide | 0.31 |
| Isopropyl alcohol | Q.S. |

Extra granular ingredients

| | |
|---|---|
| Cross linked Polyvinylpyrrolidone | 1.24 |
| Microcrystalline cellulose | 5.79 |
| Colloidal silicon dioxide | 0.12 |
| Magnesium stearate | 0.31 |

1. Fexofenadine hydrochloride, cross linked polyvinylpyrrolidone, polyvinylpyrrolidone, microcrystalline cellulose and colloidal silicon dioxide were mixed together.
2. The mixture of step 1 was granulated with isopropyl alcohol to form fexofenadine granules.
3. The granules of step 2 were mixed with the given extra granular ingredients to form fexofenadine blend.

### III. Preparation of Bilayer Tablets

| | Percent w/w of |
|---|---|
| Ingredients | tablet |
| Pseudoephedrine blend | 65.97 |
| Fexofenadine blend | 31.12 |

Film coating

| | |
|---|---|
| Opadry white | 2.91 |
| water | Q.S. |

1. The pseudoephedrine and the fexofenadine blends were compressed together into bilayered tablets.
2. The tablets were then coated with Opadry coating.

### Example 2

### I. Preparation of Pseudoephedrine Layer

| | |
|---|---|
| | Percent w/w of |
| Ingredients | tablet |

Core

| | |
|---|---|
| Microcrystalline cellulose spheres | 6.22 |
| Pseudoephedrine hydrochloride | 14.94 |
| Hydroxypropyl methyl cellulose | 1.49 |
| Purified water | Q.S |

Rate-controlling polymer coating

| | |
|---|---|
| Ethyl cellulose | 5.21 |
| Acetyl tributyl citrate | 0.52 |
| Isopropyl alcohol | Q.S |
| Purified water | Q.S |

Extra granular ingredients

| | |
|---|---|
| Polyethylene glycol | 6.82 |
| Polyvinylpyrrolidone | 2.49 |
| Microcrystalline cellulose | 27.34 |
| Colloidal silicon dioxide | 0.62 |
| Magnesium stearate | 0.31 |

1. Pseudoephedrine hydrochloride and hydroxypropyl methyl cellulose were dissolved in purified water and layered over microcrystalline cellulose spheres.
2. The cores of step 1 were coated with the rate-controlling polymer coating composition.
3. The cores of step 2 were mixed with extra granular ingredients to form pseudoephedrine blend.

### II. Preparation of Fexofenadine Layer

| | |
|---|---|
| | Percent w/w of |
| Ingredients | tablet |

Intra granular ingredients

| | |
|---|---|
| Fexofenadine hydrochloride | 11.20 |
| Cross linked Polyvinylpyrrolidone | 0.93 |
| Polyvinylpyrrolidone | 1.24 |
| Microcrystalline cellulose | 9.96 |
| Colloidal silicon dioxide | 0.31 |
| Isopropyl alcohol | Q.S. |

Extra granular ingredients

| | |
|---|---|
| Cross linked Polyvinylpyrrolidone | 1.24 |
| Microcrystalline cellulose | 5.79 |
| Colloidal silicon dioxide | 0.12 |
| Magnesium stearate | 0.31 |

1. Fexofenadine hydrochloride, cross linked polyvinylpyrrolidone, polyvinylpyrrolidone, microcrystalline cellulose and colloidal silicon dioxide were mixed together.
2. The mixture of step 1 was granulated with isopropyl alcohol to form fexofenadine granules.
3. The granules of step 2 were mixed with extra granular ingredients to form fexofenadine blend.

### III. Preparation of Bilayer Tablets

| | Percent w/w of |
|---|---|
| Ingredients | tablet |
| Pseudoephedrine blend | 65.97 |
| Fexofenadine blend | 31.12 |

Film coating

| | |
|---|---|
| Opadry white | 2.91 |
| water | Q.S. |

1. The pseudoephedrine and the fexofenadine blends were compressed together into bilayered tablets.
2. The tablets were coated with Opadry coating.

### Example 3

### I. Preparation of Pseudoephedrine Layer

| | |
|---|---|
| | Percent w/w of |
| Ingredients | tablet |

Core

| | |
|---|---|
| Microcrystalline cellulose spheres | 6.45 |
| Pseudoephedrine hydrochloride | 15.48 |
| Hydroxypropyl methyl cellulose | 1.55 |
| Purified water | Q.S |

Rate-controlling polymer coating

| | |
|---|---|
| Ethyl cellulose | 5.40 |
| Acetyl tributyl citrate | 0.54 |
| Isopropyl alcohol | Q.S |
| Purified water | Q.S |

Extra granular ingredients

| | |
|---|---|
| Polyethylene glycol | 6.45 |
| Polyvinylpyrrolidone | 2.58 |
| Microcrystalline cellulose | 28.31 |
| Colloidal silicon dioxide | 0.64 |
| Magnesium stearate | 0.32 |

1. Pseudoephedrine hydrochloride and hydroxypropyl methyl cellulose were dissolved in purified water and layered over microcrystalline cellulose spheres.
2. The cores of step 1 were coated with the rate-controlling polymer coating composition.
3. The cores of step 2 were mixed with extra granular ingredients to form pseudoephedrine blend.

### II. Preparation of Fexofenadine Layer

| | |
|---|---|
| | Percent w/w of |
| Ingredients | tablet |

Intra granular ingredients

| | |
|---|---|
| Fexofenadine hydrochloride | 11.61 |
| Cross linked Polyvinylpyrrolidone | 0.97 |
| Polyvinylpyrrolidone | 1.29 |
| Microcrystalline cellulose | 10.32 |
| Colloidal silicon dioxide | 0.16 |
| Magnesium stearate | 0.16 |

Extra granular ingredients

| | |
|---|---|
| Cross linked Polyvinylpyrrolidone | 1.29 |
| Microcrystalline cellulose | 6.32 |
| Magnesium stearate | 0.16 |

1. Fexofenadine hydrochloride, cross linked polyvinylpyrrolidone, polyvinylpyrrolidone, microcrystalline cellulose and colloidal silicon dioxide were mixed together.
2. The mixture of step 1 was compacted and milled to form fexofenadine granules.
3. The granules of step 2 were mixed with extra granular ingredients to form fexofenadine blend.

### III. Preparation of Bilayer Tablets

| | Percent w/w of |
|---|---|
| Ingredients | tablet |
| Pseudoephedrine blend | 67.72 |
| Fexofenadine blend | 32.28 |

The pseudoephedrine and the fexofenadine blends were compressed together into bilayered tablets.

### Example 4

### I. Preparation of Pseudoephedrine Layer

| | |
|---|---|
| | Percent w/w of |
| Ingredients | tablet |

Core

| | |
|---|---|
| Microcrystalline cellulose spheres | 6.23 |
| Pseudoephedrine hydrochloride | 14.95 |
| Hydroxypropyl methyl cellulose | 1.49 |
| Purified water | Q.S |

Rate-controlling polymer coating

| | |
|---|---|
| Ethyl cellulose | 5.15 |
| Acetyl tributyl citrate | 0.52 |
| Isopropyl alcohol | Q.S |
| Purified water | Q.S |

Cushioning agent coating

| | |
|---|---|
| Polyethylene glycol 6000 | 2.83 |
| Isopropyl alcohol | Q.S |
| Purified water | Q.S |

Extra granular ingredients

| | |
|---|---|
| Polyethylene glycol 6000 | 3.99 |
| Polyvinylpyrrolidone | 2.49 |
| Microcrystalline cellulose | 27.36 |
| Colloidal silicon dioxide | 0.62 |
| Magnesium stearate | 0.31 |

1. Pseudoephedrine hydrochloride and hydroxypropyl methyl cellulose were dissolved in purified water and layered over microcrystalline cellulose spheres.
2. The cores of step 1 were coated with the rate-controlling polymer coating composition.
3. The cores of step 2 were coated with the cushioning agent coating composition.
4. The cores of step 3 were mixed with the extra granular ingredients to form pseudoephedrine blend.

### II. Preparation of Fexofenadine Layer

| | |
|---|---|
| | Percent w/w of |
| Ingredients | tablet |

Intra granular ingredients

| | |
|---|---|
| Fexofenadine hydrochloride | 11.21 |
| Cross linked Polyvinylpyrrolidone | 0.93 |
| Polyvinylpyrrolidone | 1.25 |
| Microcrystalline cellulose | 9.97 |
| Colloidal silicon dioxide | 0.31 |
| Isopropyl alcohol | Q.S. |

Extra granular ingredients

| | |
|---|---|
| Cross linked Polyvinylpyrrolidone | 1.25 |
| Mannitol | 5.79 |
| Colloidal silicon dioxide | 0.12 |
| Magnesium stearate | 0.31 |

1. Fexofenadine hydrochloride, cross linked polyvinylpyrrolidone, polyvinylpyrrolidone, microcrystalline cellulose and colloidal silicon dioxide were mixed together.
2. The mixture of step 1 was granulated with isopropyl alcohol to form fexofenadine granules.
3. The granules of step 2 were mixed with the given extra granular ingredients to form fexofenadine blend.

### III. Preparation of Bilayer Tablets

| | Percent w/w of |
|---|---|
| Ingredients | tablet |
| Pseudoephedrine blend | 65.95 |
| Fexofenadine blend | 31.14 |

Film coating

| | |
|---|---|
| Opadry white | 2.91 |
| water | Q.S. |

1. The pseudoephedrine and the fexofenadine blends were compressed together into bilayered tablets.
2. The tablets were then coated with Opadry coating.

### Example 5

### Preparation of Tablets

| | |
|---|---|
| Ingredients | Percent w/w of tablet |

Core

| | |
|---|---|
| Pseudoephedrine hydrochloride | 30.39 |
| Microcrystalline cellulose | 2.53 |
| Polyvinylpyrrolidone | 2.53 |
| Hydroxypropyl methyl cellulose | 25.32 |
| Isopropyl alcohol | Q.S |
| Magnesium stearate | 0.63 |
| Colloidal silicon dioxide | 0.32 |
| Talc | 0.32 |

Seal coating

| | |
|---|---|
| Hydroxypropyl methyl cellulose | 1.13 |
| Polyethylene glycol 400 | 0.11 |
| Isopropyl alcohol | Q.S |
| Purified water | Q.S |

Polymer coating

| | |
|---|---|
| Ethyl cellulose | 5.31 |
| Polyvinylpyrrolidone | 1.86 |
| Acetyl tributyl citrate | 1.06 |
| Isopropyl alcohol | Q.S |
| Dichloromethane | Q.S |

Fexofenadine Drug layering

| | |
|---|---|
| Fexofenadine hydrochloride | 22.79 |
| Polyvinylpyrrolidone | 1.27 |
| Crosscarmellose sodium | 1.90 |
| Lactose | 2.53 |
| Isopropyl alcohol | Q.S |

1. Pseudoephedrine hydrochloride, polyvinylpyrrolidone, microcrystalline cellulose, and hydroxypropyl methyl cellulose were mixed together.
2. The mixture of step 1 was granulated with isopropyl alcohol to form fexofenadine granules.
3. The granules of step 2 were mixed with magnesium stearate, colloidal silicon dioxide and talc and compressed into tablets.
4. The tablets of step 3 were coated with the seal coating composition.
5. The coated tablets of step 4 were coated with the rate-controlling polymer coating composition.
6. The coated tablets of step 5 were then coated with the Fexofenadine drug layering composition.

### Example 6

### Dissolution Profile of Allegra D-24 vs Test Tablet

The dissolution of the formulation prepared according to Example 4 and Allegra D-24 were carried out in 900 ml of 0.001NHCl using USP Apparatus II at 50 RPM, 37° C.+0.5° C.

### Percent Drug Release of Fexofenadine Hydrochloride

| | 10 min | 20 min | 30 min | 45 min | 60 min | 120 min |
|---|---|---|---|---|---|---|
| Allegra D-24 | 56 | 81 | 85 | 88 | 89 | 91 |
| Test tablet | 38 | 51 | 75 | 77 | 78 | 82 |

### Percent Drug Release of Pseudoephedrine Hydrochloride

| | | | | | | 11 | | 15 | | 23 |
|---|---|---|---|---|---|---|---|---|---|---|
| | 1 hr | 2 hr | 3 hr | 7 hr | 9 hr | hr | 13 hr | hr | 17 hr | hr |
| Allegra | 1 | 8 | 17 | 46 | 59 | 69 | 77 | 82 | 85 | 90 |
| D-24 | | | | | | | | | | |
| Test | 5 | 15 | 27 | 58 | 66 | 72 | 76 | 80 | 83 | 89 |
| tablet | | | | | | | | | | |

### Example 7

### Dissolution Profile of Allegra D-24 vs Test Tablet

The dissolution of the formulation prepared according to Example 5 and Allegra D-24 were carried out in 900 ml of 0.001NHCl using USP Apparatus II at 50 RPM, 37° C.+0.5° C.

### Percent Drug Release of Fexofenadine Hydrochloride

| | 10 min | 20 min | 30 min | 45 min | 60 min | 120 min |
|---|---|---|---|---|---|---|
| Allegra D-24 | 56 | 81 | 85 | 88 | 89 | 91 |
| Test tablet | 10 | 18 | 22 | 28 | 35 | 50 |

### Percent Drug release of Pseudoephedrine Hydrochloride

| | 1 hr | 2 hr | 3 hr | 7 hr | 9 hr | 11 hr | 15 hr | 19 hr | 23 hr |
|---|---|---|---|---|---|---|---|---|---|
| Allegra | 1 | 8 | 17 | 46 | 59 | 69 | 82 | 87 | 90 |
| D-24 | | | | | | | | | |
| Test | 0 | 3 | 9 | 36 | 51 | 66 | 86 | 101 | 107 |
| tablet | | | | | | | | | |

While several particular forms of the invention have been illustrated and described, it will be apparent that various modifications and combinations of the invention detailed in the text can be made without departing from the spirit and scope of the invention.

We claim:

1. A layered composition of an antihistamine and a decongestant comprising:
(i) an antihistamine layer comprising:
(a) therapeutically effective amount of an antihistamine or its pharmaceutically effective salts; and
(b) one or more pharmaceutically acceptable excipients;
(ii) a decongestant layer comprising:
(a) a core comprising therapeutically effective amount of decongestant or its pharmaceutically effective salts;
(b) a coating over the core comprising one or more rate-controlling polymers; and
(c) one or more pharmaceutically acceptable excipients.

2. A layered composition according to claim 1, wherein the core is embedded in a carrier matrix.

3. A layered composition according to claim 1, wherein the antihistamine comprises a H1 antagonist.

4. A layered composition according to claim 3, wherein the antihistamine is comprises fexofenadine, loratadine, cetirizine, terfenadine, acrivastine, astemizole, and its pharmaceutically acceptable salts, solvates, polymorphs, enantiomers, single isomer, or mixtures thereof.

5. A layered composition according to claim 1, wherein the decongestant is comprises pseudoephedrine, oxymetazoline, phenylephrine, xylometazoline, and its pharmaceutically acceptable salts, solvates, polymorphs, enantiomers, single isomer, or mixtures thereof.

6. A layered composition according to claim 1, wherein the antihistamine is fexofenadine and the decongestant is pseudoephedrine.

7. A layered composition according to claim 1, wherein the decongestant layer may further comprises one or more cushioning agents.

8. A layered composition according to claim 7, wherein the cushioning agent comprises one or more of waxes, fats, lipids, polyethylene glycols, polyoxyethylenes, or gums or mixture thereof.

9. A layered composition according to claim 1, wherein the rate-controlling polymers used to provide coating over decongestant core comprises one or more of hydrophilic polymers, hydrophobic polymers or combinations thereof.

10. A layered composition according to claim 1, wherein the hydrophilic rate controlling polymer comprises one or more of cellulose derivatives, polyvinylpyrrolidone, polyvinyl acetate, copolymer of vinylpyrrolidone and vinyl acetate, polysaccharides, polyalkylene glycols, starch, gums and derivatives or mixtures thereof.

11. A layered composition according to claim 1, wherein the hydrophobic rate controlling polymer comprises one or more of ethyl cellulose, cellulose acetate, cellulose acetate butyrate, hydroxypropyl methylcellulose phthalate, poly (alkyl)methacrylate, and copolymers of acrylic or methacrylic acid esters, waxes, shellac or hydrogenated vegetable oils or mixtures thereof.

12. A layered composition according to claim 1, wherein the excipients comprises one or more of fillers, binders, disintegrants, lubricants, glidants, colorants or flavoring agents or mixtures thereof.

13. A process for preparing the layered composition comprising the steps of:
(i) providing an antihistamine layer by:
(a) preparing granules comprising an antihistamine or its pharmaceutically effective salts with one or more pharmaceutically acceptable excipients; and
(b) combining the granules of antihistamine with one or more pharmaceutically acceptable excipients;
(ii) providing a decongestant layer by:
(a) preparing cores comprising a decongestant or its pharmaceutically effective salts;
(b) coating the cores with one or more rate-controlling polymers; and
(c) combining the coated cores of decongestant with one or more pharmaceutically acceptable excipients; and
(iii) compressing the antihistamine layer and the decongestant layer into layered composition.

14. A process for preparing the layered composition comprising the steps of:
(i) providing an antihistamine layer by:
(a) preparing a solution, suspension or dispersion comprising an antihistamine or its pharmaceutically effective salts with one or more pharmaceutically acceptable excipients;
(ii) providing a decongestant layer by:
(a) preparing cores comprising a decongestant or its pharmaceutically effective salts;
(b) optionally coating the cores with a seal coating;
(c) coating the cores with a coating comprising one or more rate-controlling polymers;
(iii) layering the antihistamine layer over the decongestant layer to form a layered composition.

15. A method for treating nasal congestion or allergy-related disorders by administering a layered composition of an antihistamine and a decongestant comprising:
(i) an antihistamine layer comprising:
(a) therapeutically effective amount of an antihistamine or its pharmaceutically effective salts; and
(b) one or more pharmaceutically acceptable excipients;
(ii) a decongestant layer comprising:
(a) a core comprising therapeutically effective amount of decongestant or its pharmaceutically effective salts;
(b) a coating over the core comprising one or more rate-controlling polymers; and
(c) one or more pharmaceutically acceptable excipients.
